(19) United States
(12) Patent Application Publication
(10) Pub. No.: US 2011/0112412 A1
(43) Pub. Date: May 12, 2011

(54) CUFF FOR BLOOD PRESSURE INFORMATION MEASURING DEVICE AND BLOOD PRESSURE INFORMATION MEASURING DEVICE EQUIPPED WITH THE SAME

(75) Inventors: Yoshihiko Sano, Kyoto (JP); Hiroyasu Ariga, Kyoto (JP); Chisato Uesaka, Kyoto (JP); Yoshihide Onishi, Dalian (CN); Takahide Tanaka, Shiga (JP)

(73) Assignee: OMRON HEALTHCARE CO., LTD., Kyoto (JP)

(21) Appl. No.: 13/002,431

(22) PCT Filed: Jun. 18, 2009
(86) PCT No.: PCT/JP2009/061080

§ 371 Date: Jan. 3, 2011

(30) Foreign Application Priority Data
Jul. 7, 2008 (JP) 2008-177098

(51) Int. Cl. A61B 5/022

(52) U.S. Cl. 600/499

(57) ABSTRACT

A cuff for a blood pressure information measuring device configured to be mounted on a measurement site for measuring blood pressure information has a fluid bag for compressing the measurement site, a flexible curved elastic plate located outside the fluid bag while the fluid bag is wrapped around the measurement site, and annular or arc-shaped to extend along the measurement site, an inflation and deflation mechanism secured to the curved elastic plate and inflating and deflating the fluid bag, and a bag-shaped cover body encapsulating the fluid bag, the inflation and deflation mechanism, and the curved elastic plate.

### BACKGROUND OF THE INVENTION

1. Technical Field

The present invention relates to a cuff for a blood pressure information measuring device that is mounted on a site for measurement in use, and a blood pressure information measuring device equipped with the cuff.

2. Background Art

It is highly important to acquire information about the blood pressure of a subject in order to know the health condition of the subject. In recent years, not only the systolic pressure value, the diastolic pressure value and the like which have widely been recognized as useful as representative indices for healthcare, but also the pulse wave of the subject is taken in an attempt for example to identify a change in heart load and hardness of the artery. A blood pressure information measuring device is an instrument for obtaining these indices for healthcare based on acquired information about blood pressure, and is being expected to be further applied to such fields as early detection, prevention, treatment and the like of circulatory system diseases. The aforementioned blood pressure information includes a wide variety of information about the circulatory system such as systolic pressure value, diastolic pressure value, mean blood pressure value, pulse wave, pulse rate, and AI (Augumentation Index).

Generally, the blood pressure information is measured with a cuff for a blood pressure information measuring device that has a fluid bag therein (hereinafter also referred to as cuff simply). Here, a cuff refers to a band-like structure that has an inner cavity and can be wrapped around a part of a living body, and a fluid such as gas or liquid is injected into the inner cavity so that the fluid bag is inflated and deflated for use in measurement of the blood pressure information. For example, regarding a blood pressure information measuring device (hereinafter also referred to as blood pressure monitor simply) for measuring blood pressure values such as systolic pressure value and diastolic pressure value, a cuff having a fluid bag therein for compressing the artery is wrapped around a surface of a living body, and the wrapped fluid bag is inflated and deflated to acquire the artery pressure pulse wave as a change of the internal pressure of the fluid bag and thereby measure the blood pressure value. In particular, a cuff of the type wrapped around an arm in use is called arm belt or manchette.

Usually, for measurement of the blood pressure information, an upper arm or wrist is used as a site for measurement. The cuff is therefore wrapped around an upper arm or wrist in use. The above-described blood pressure monitors are classified by the difference in site for measurement into an upper arm blood pressure monitor and a wrist blood pressure monitor.

For the wrist blood pressure monitor, generally a structure in which the cuff and a main body are integrated is employed. More specifically, on the outer peripheral surface of the cuff having therein an air bag as the fluid bag, a casing to serve as a main body is attached. In the casing, various kinds of components such as an inflation and deflation mechanism for inflating and deflating the air bag (generally pressurizing pump, exhaust valve and the like) are housed. This is because the circumferential length of the wrist to be used as a measurement site is shorter than the upper arm and the artery to be compressed is located at a relatively shallow position from the body surface, and therefore, the air bag may be of a small capacity and accordingly the inflation and deflation mechanism of a relatively small output and a small size can be used, and the components can be accommodated in the small-sized casing.

In contrast, as for the upper arm blood pressure monitor, because the circumferential length of the upper arm to be used as a measurement site is longer than the wrist and the artery to be compressed is located at a relatively deep position from the body surface, and therefore, the air bag is required to have a large capacity and accordingly the inflation and deflation mechanism of a high output and a large size is necessary. Therefore, regarding the upper arm blood pressure monitor, generally the cuff and the main body are not integrated but configured as separate and independent components.

Some upper arm blood pressure monitors, however, are configured so that the cuff and the main body are integrated. Such a blood pressure monitor having the integrated cuff and main body is intended to be used in such a manner that the main body is mounted on a table or the like, and has a structure in which the main body is provided with various kinds of components such as inflation and deflation mechanism, the main body has a hollow opening formed to allow a measurement site to be inserted in the opening, and the cuff containing an air bag is provided to extend along the hollow opening of the main body. As for the blood pressure monitor configured in the above-described manner, the main body in use must be mounted on a mount surface such as table, and therefore, such a blood pressure monitor can be only used under a limited environment for use, and is not necessarily excellent in terms of convenience for use.

Meanwhile, recently various kinds of components such as the inflation and deflation mechanism as described above have been remarkably downsized, and components of high output and small size have been prevailed. Therefore, in the upper-arm blood pressure monitor as well, such high-output and small-size components can be used to considerably downsize the main body as compared with the conventional one. Accordingly, for an upper-arm blood pressure monitor configured to have a cuff and a main body separate from each other as well, a structure is becoming possible in which various kinds of components such as inflation and deflation mechanism are accommodated in a small-sized casing and the casing is integrated with the cuff.

Some documents note such downsizing of various kinds of components and disclose a blood pressure monitor for which integration of a small-sized main body with a cuff is studied. Such documents include for example Japanese Patent Laying-Open No. 3-85138 (Patent Document 1), Japanese Patent Laying-Open No. 2000-83912 (Patent Document 2), and Japanese Patent Laying-Open No. 7-163531 (Patent Document 3).

A blood pressure monitor disclosed in above-referenced Japanese Patent Laying-Open No. 3-85138 is configured in such a manner that a cuff and a main body are formed separately, various kinds of components such as inflation and deflation mechanism are accommodated in the main body, and the main body can be attached removably at any position on the outer peripheral surface of the cuff. A blood pressure monitor disclosed in above-referenced Japanese Patent Laying-Open No. 2000-83912 is configured in such a manner that a cuff and a main body are formed separately, the main body can be attached removably to a base portion provided on the outer peripheral surface of the cuff, various kinds of components such as inflation and deflation mechanism are provided to the base portion, and only a display portion and an operation portion are provided on the main body. Further, a blood pressure monitor disclosed in above-referenced Japanese Patent Laying-Open No. 7-163531 is configured in such a manner that an opening is formed at a predetermined position of the outer peripheral surface of a bag-like cover body extending over the surface of the cuff, a part of a casing which is a main body is embedded inside the bag-like cover body to close the opening, and various kinds of components such as inflation and deflation mechanism are accommodated in the partially embedded casing.

These blood pressure monitors disclosed in Japanese Patent Laying-Open Nos. 3-85138, 2000-83912, and 7-163531 are each configured so that the monitor can take a measurement without mounting the main body on a mount surface such as table, and thus the blood pressure monitors are of small size suitable for being carried.
Patent Document 1: Japanese Patent Laying-Open No. 3-85138
Patent Document 2: Japanese Patent Laying-Open No. 2000-83912
Patent Document 3: Japanese Patent Laying-Open No. 7-163531

### SUMMARY OF THE INVENTION

In the case where the structure as disclosed in above-referenced Japanese Patent Laying-Open No. 3-85138 is employed, an air tube connecting the cuff and the main body may hinder measurement, and handling of the air tube in storage can be difficult. In the case where the structure as disclosed in above-referenced Japanese Patent Laying-Open No. 2000-83912 is employed, the base portion is thick, convenience for use may be wanting, and the blood pressure monitor may therefore be unsuitable for being carried. In the case where the structure as disclosed in above-referenced Japanese Patent Laying-Open Nos. 2000-83912 and 7-163531 is employed the portion of the cuff on which the main body is provided while the cuff is mounted on a measurement site (the main body here is the base portion in Japanese Patent Laying-Open No. 2000-83912 and is the casing in Japanese Patent Laying-Open No. 7-163531) does not flexibly deform, and the cuff does not easily fit the measurement site, so that a gap is generated between the measurement site and the cuff, and the accuracy in measurement of the blood pressure value is deteriorated.

In the case as well where the wrist blood pressure monitor is configured such that the casing is attached to the outer peripheral surface of the cuff as is conventionally employed the casing is thick, the convenience for use may be wanting, and the blood pressure monitor may therefore be unsuitable for being carried, like the structure disclosed in above-referenced Japanese Patent Laying-Open No. 2000-83912.

One or more embodiments of the invention provides a blood pressure information measuring device that can be downsized and is excellent in handling and portability, as well as a cuff for the blood pressure information measuring device to be equipped with the cuff.

A cuff for a blood pressure information measuring device according to one or more embodiments of the present invention is mounted in use on a measurement site for measuring blood pressure information, and includes: a fluid bag for compressing the measurement site; a flexible curved elastic plate located outside the fluid bag while the fluid bag is wrapped around the measurement site, and annular or arc-shaped to extend along the measurement site; an inflation and deflation mechanism secured to the curved elastic plate and inflating and deflating the fluid bag; and a bag-shaped cover body encapsulating the fluid bag, the inflation and deflation mechanism, and the curved elastic plate.

Regarding the cuff for a blood pressure information measuring device according to one or more embodiments of the present invention, the inflation and deflation mechanism is disposed on a main surface of the curved elastic plate, the main surface being opposite to a main surface of the curved elastic plate that faces the fluid bag.

Regarding the cuff for a blood pressure information measuring device according to one or more embodiments of the present invention, the cuff further includes a protection member covering an exposed surface of the inflation and deflation mechanism.

Regarding the cuff for a blood pressure information measuring device according to one or more embodiments of the present invention, the inflation and deflation mechanism includes a pressurizing pump and an exhaust valve.

Regarding the cuff for a blood pressure information measuring device according to one or more embodiments of the present invention, the cuff may further include a power supply unit supplying electric power for driving the inflation and deflation mechanism, and a power supply switch. According to one or more embodiments of the present invention, the power supply unit is encapsulated in the bag-shaped cover body.

Regarding the cuff for a blood pressure information measuring device according to one or more embodiments of the present invention, the cuff may further include a pressure detection unit for detecting an internal pressure of the fluid bag. According to one or more embodiments of the present invention, the pressure detection unit is encapsulated in the bag-shaped cover body.

A blood pressure information measuring device according to one or more embodiments of the present invention includes: a cuff for the blood pressure information measuring device according to one or more embodiments of the present invention as described above, and an associated unit provided separately and independently of the cuff for the blood pressure information measuring device, and the associated unit is provided with a display portion for displaying blood pressure information as a result of measurement.

Regarding the blood pressure information measuring device according to one or more embodiments of the present invention, the associated unit is further provided with an operation portion for entering an instruction to drive the inflation and deflation mechanism.

The blood pressure information measuring device according to one or more embodiments of the present invention further includes a communication unit enabling radio communication to be performed between the cuff for the blood pressure information measuring device and the associated unit.

According to one or more embodiments of the present invention, a blood pressure information measuring device that can be downsized and is excellent in handling and portability as well as a cuff for the blood pressure information measuring device to be equipped with the cuff can be provided.

### BRIEF DESCRIPTION OF THE DRAWINGS

FIG. 1 is a perspective view showing an external structure of a blood pressure monitor in a first embodiment of the present invention.

FIG. 2 is a block diagram showing a configuration of functional blocks of the blood pressure monitor shown in FIG. 1.

FIG. 3 is a schematic cross section of a cuff unit along line shown in FIG. 1.

FIG. 4 is a perspective view where an exterior cover of the cuff unit shown in FIG. 1 is removed.

FIG. 5 is a schematic diagram showing a posture to be taken of a subject for the blood pressure monitor in the first embodiment of the present invention.

FIG. 6 is a flowchart showing a flow of a process of measuring the blood pressure value with the blood pressure monitor in the first embodiment of the present invention.

FIG. 7 is a cross section of a cuff unit showing a modification of the blood pressure monitor in the first embodiment of the present invention.

FIG. 8 is a perspective view showing an external structure of a blood pressure monitor in a second embodiment of the present invention.

FIG. 9 is a schematic cross section of a cuff unit of the blood pressure monitor in the second embodiment of the present invention.

FIG. 10 is a perspective view showing an external structure of a blood pressure monitor in a third embodiment of the present invention.

FIG. 11 is a block diagram showing a configuration of functional blocks of the blood pressure monitor shown in FIG. 10.

FIG. 12 is a perspective view showing a detailed structure of a cuff unit shown in FIG. 10.

FIG. 13 is a schematic cross section of a cuff along line XIII-XIII shown in FIG. 12.

FIG. 14 is a schematic top view showing a structure of a tightening length adjustment mechanism shown in FIG. 12.

FIG. 15 is a flowchart showing a process procedure of the blood pressure monitor in the third embodiment of the present invention.

FIG. 16 is a timing chart showing in time series manner an operational condition or operational state of each component of the blood pressure monitor in the third embodiment of the present invention.

FIG. 17 is a schematic diagram for illustrating a mount operation of mounting the cuff unit in the third embodiment of the present invention on an upper arm.

FIG. 18 is a schematic diagram of a mount state where the cuff unit in the third embodiment of the present invention is mounted on an upper arm.

### DETAILED DESCRIPTION

In the following, embodiments of the present invention will be described in detail with reference to the drawings. In embodiments of the invention, numerous specific details are set forth in order to provide a more thorough understanding of the invention. However, it will be apparent to one of ordinary skill in the art that the invention may be practiced without these specific details. In other instances, well-known features have not been described in detail to avoid obscuring the invention. In connection with the embodiments illustrated below, as a cuff for a blood pressure information measuring device and a blood pressure information measuring device provided with the cuff, a cuff for a blood pressure monitor that is intended to be wrapped around an upper arm in use and a blood pressure monitor provided with the cuff, respectively, will exemplarily be described.

### First Embodiment

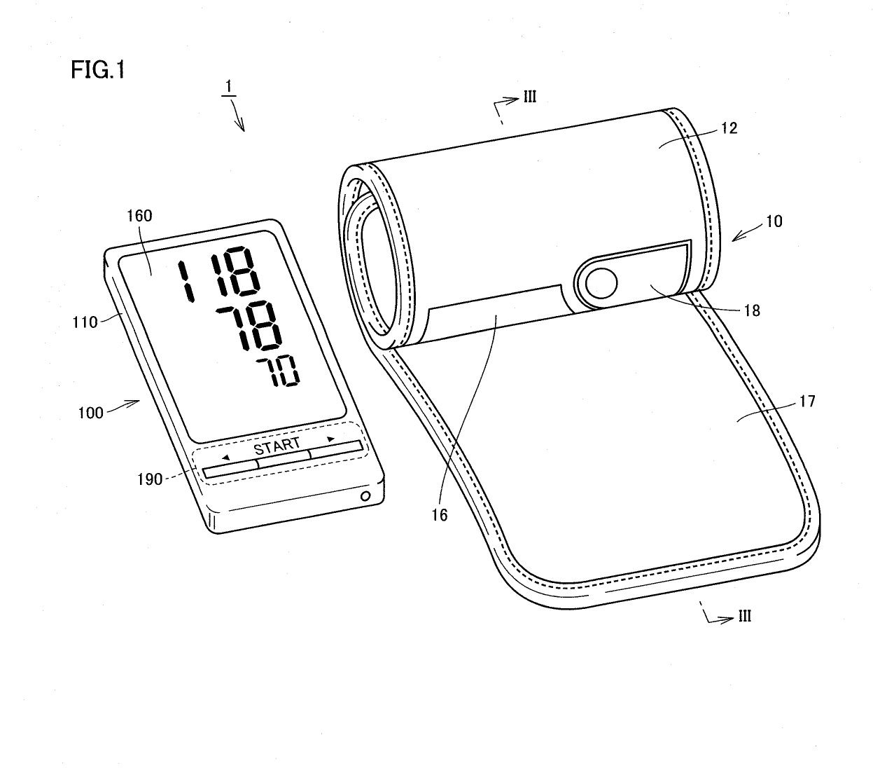

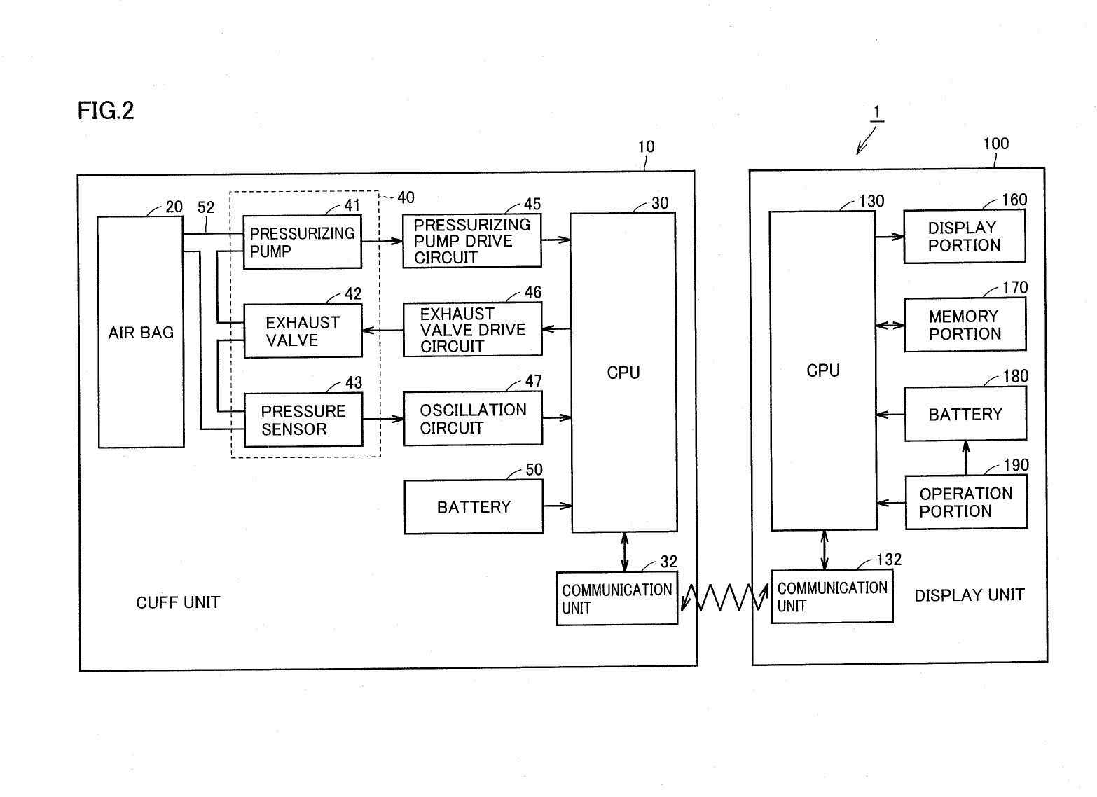

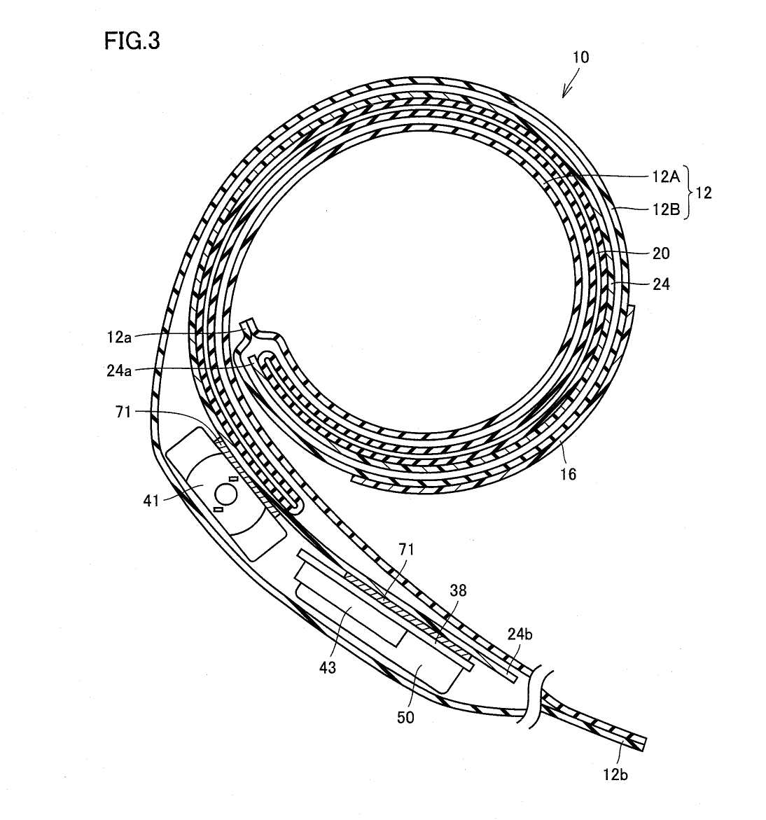

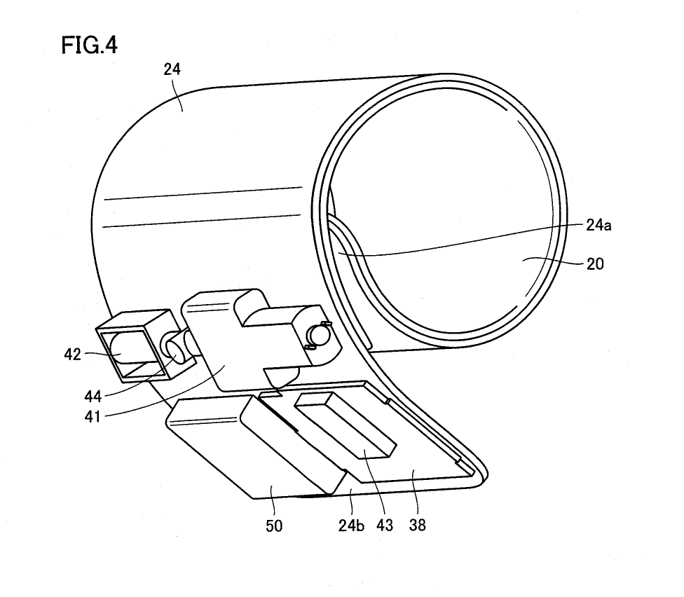

FIG. 1 is a perspective view showing an external structure of a blood pressure monitor in a first embodiment of the present invention. FIG. 2 is a block diagram showing a configuration of functional blocks of the blood pressure monitor shown in FIG. 1. FIG. 3 is a schematic cross section of a cuff unit along line shown in FIG. 1. FIG. 4 is a perspective view where an exterior cover of the cuff unit shown in FIG. 1 is removed. Referring first to FIGS. 1 to 4, a structure of the blood pressure monitor and a cuff for the blood pressure monitor to be equipped with the cuff will be described.

As shown in FIG. 1, blood pressure monitor 1 in the present embodiment includes a cuff unit 10 and a display unit 100 that are configured separately and independently of each other. Cuff unit 10 is configured in an annular form having a cut opened at a predetermined position in the circumferential direction and extending in the axial direction so that the cuff unit can be wrapped around an upper arm. In contrast, display unit 100 has a low-profile casing 110 that is substantially in the form of a rectangular parallelepiped, and has a top surface provided with a display portion 160 and an operation portion 190. Cuff unit 10 corresponds to the above-described cuff for a blood pressure monitor, and display unit 100 corresponds to an associated unit associated with cuff unit 10.

As shown in FIGS. 2 to 4, cuff unit 10 includes an air bag 20 to serve as a fluid bag for compressing an upper arm, a curler 24 to serve as a curved elastic plate to be located outside air bag 20 where cuff unit 10 is mounted on the upper arm, and an exterior cover 12 to serve as a bag-shaped cover body encapsulating these air bag 20 and curler 24.

According to one or more embodiments of the present invention, an air bag 20 is formed of a bag-shaped member produced using resin sheets, and has an inflation and deflation space in the air bag. As air bag 20, for example, a component is used that is formed in the shape of a bag by superposing two resin sheets on each other and welding respective perimeters together. The inflation and deflation space of air bag 20 is connected through an air tube 52, which is described later, to a pressurizing pump 41 and an exhaust valve 42, which are also described later, and these pressurizing pump 41 and exhaust valve 42 are used to increase and decrease the pressure of the space. As to the material for the resin sheets constituting air bag 20, any material can be used as long as the material is highly stretchable and accompanied by no air leakage from the inflation and deflation space after the sheets are welded. In view of this, according to one or more embodiments of the present invention, a possible material for the resin sheets includes ethylene vinyl acetate (EVA), soft polyvinyl chloride (PVC), polyurethane (PU), polyamide (PA), raw rubber, and the like.

Curler 24 is formed of a flexible member configured to be elastically deformable in the radial direction by being annularly wrapped, and has an inner end 24a and an outer end 24b with respect to the longitudinal direction. Curler 24 is attached and secured to the outer peripheral surface of air bag 20 with an adhesive member such as double-sided tape (not shown), and is configured to be located along the upper arm by maintaining the annular form of the curler itself. This curler 24 aims to allow a subject to easily mount cuff unit 10 on the upper min and to bias air bag 20 toward the upper arm while cuff unit 10 is mounted on the upper arm. Curler 24 is formed using a resin member such as polypropylene (PP) for example so that the curler exhibits an adequate elastic force.

Exterior cover 12 includes an inner cover 12A which when mounted contacts the surface of the upper arm and an outer cover 12B which when mounted is located at an outermost position. The inner cover and the outer cover are superposed on each other and respective perimeters are joined (by sewing, welding or the like for example) so that exterior cover 12 is formed in the shape of a bag. Exterior cover 12 has an inner end 12a and an outer end 12b with respect to the longitudinal direction. On the outer peripheral surface located near inner end 12a of exterior cover 12 and the inner peripheral surface located near outer end 12b thereof, hook-and-loop fastener parts 16 and 17 (see FIG. 1) are provided, respectively. The portion near inner end 12a and the portion near outer end 12b are laid on each other on the surface of the upper arm, and hook-and-loop fastener parts 16 and 17 engage each other, so that cuff unit 10 is wrapped around and secured to the upper arm.

According to one or more embodiments of the present invention, for the inner cover 12A of exterior cover 12, a sufficiently stretchable member is used so that a compressing force resulting from inflation of air bag 20 and applied to the upper arm is not counteracted by inner cover 12A. In contrast, for outer cover 12B, a member less stretchable relative to inner cover 12A is used. In view of this, for exterior cover 12, cloth or the like constituted of synthetic fibers of polyamide (PA), polyester or the like having its stretchability that can be adjusted relatively easily is used.

Further, as shown in FIG. 1, at a predetermined position of the outer peripheral surface of exterior cover 12, a mark 18 is provided that indicates a position to be held with a hand different from the aim on which cuff unit 10 is mounted, when cuff unit 10 is to be mounted on the upper arm. More specifically, mark 18 is formed of a member made of an elastomer and having a recess which is formed on its surface and on which a thumb is to be placed, and is attached at a predetermined position to outer cover 12B of exterior cover 12.

As shown in FIGS. 2 to 4, cuff unit 10 further includes pressurizing pump 41 delivering air to air bag 20 and thereby inflating air bag 20, exhaust valve 42 releasing the air from air bag 20 and thereby deflating air bag 20, a pressure sensor 43 to serve as a pressure detection unit, air tube 52 including an air joint 44, a circuit board 38 on which various types of electrical circuits are formed, and a battery 50 to serve as a power supply unit. Of these components, pressure sensor 43 is mounted on circuit board 38, and pressurizing pump 41, exhaust valve 42, air joint 44, circuit board 38, and battery 50 are all arranged on the outer peripheral surface of curler 24 (namely on a main surface of curler 24 that is opposite to the main surface (inner peripheral surface) thereof facing air bag 20). The above-described various kinds of components are therefore all encapsulated in exterior cover 12.

More specifically, as shown in FIG. 3, pressurizing pump 41, exhaust valve 42, circuit board 38, and battery 50 are all secured to curler 24 with an adhesive sheet 71 to serve as a securing member interposed therebetween. Here, curler 24 is curved to extend along the upper arm as described above, and therefore, according to one or more embodiments of the present invention, the adhesive sheet 71 to be used is an adhesive sheet including a base formed of a cushion member such as sponge member, rubber member, or resin member, and adhesive layers provided on respective sides opposite to each other. Such adhesive sheet 71 can be used to fill a space between curler 24 and above-described various kinds of components by deformation of the base of adhesive sheet 71, and more stably secure the above-described various kinds of components to curler 24.

As to securing of the above-described various kinds of components to curler 24, other than the securing with the adhesive sheet, securing in a different way such as securing with a screw, or securing with a hook or the like provided on curler 24 is applicable. As to the position on curler 24 at which the above-described various kinds of components are each secured, according to one or more embodiments of the present invention, a portion of a small curvature is selected so that the secured state is more stably kept. Therefore, in cuff unit 10 of the present embodiment, those various kinds of components are secured not to the portion of curler 24 forming the hollow opening in which the upper arm is inserted, but to the portion of the curler located outside the region where a part of the curler overlaps another part of the curler.

As shown in FIG. 2, pressurizing pump 41, exhaust valve 42, and pressure sensor 43 are each connected to air bag 20 through air tube 52 including air joint 44. Air joint 44 is a branch tube for connecting respective parts of the air tube connected to these pressurizing pump 41, exhaust valve 42, and pressure sensor 43. Pressurizing pump 41 is used for supplying air into the inflation and deflation space which is a space in air bag 20, and the operation of the pump is controlled by a pressurizing pump drive circuit 45 described later. Exhaust valve 42 is used for keeping the pressure of the inflation and deflation space of air bag 20 (the pressure is hereinafter also referred to as “cuff pressure”), and opening the inflation and deflation space of air bag 20 to the outside, and the operation of the valve is controlled by an exhaust valve drive circuit 46 as described later. Pressure sensor 43 is used for detecting the internal pressure of the inflation and deflation space of air bag 20 through air tube 52, and inputs an output signal according to the detected pressure to an oscillation circuit 47 as described later. Pressurizing pump 41, exhaust valve 42, and pressure sensor 43 correspond to an air system component 40 for controlling and detecting the inflation or deflation state of air bag 20, and pressurizing pump 41 and exhaust valve 42 correspond to an inflation and deflation mechanism.

On a surface of circuit board 38, various kinds of electronics are mounted. These electronics are electrically connected through an interconnection pattern formed on the surface of circuit board 38, and accordingly various kinds of electrical circuits as described later are formed. Further, battery 50 serves as a power supply for supplying electric power to various kinds of electronics including the above-described inflation and deflation mechanism, and according to one or more embodiments of the present invention, a repeatedly rechargeable battery is used. At a predetermined position of the surface of exterior cover 12, a connection terminal for charging battery 50 and a power supply switch for operating the electrical circuits in cuff unit 10 are provided, which is not shown in FIG. 1.

As shown in FIG. 2, cuff unit 10 includes, in addition to the above-described components, a CPU (Central Processing Unit) 30, pressurizing pump drive circuit 45, exhaust valve drive circuit 46, oscillation circuit 47, and a communication unit 32. CPU 30 is a means for controlling the whole of blood pressure monitor 1. Pressurizing pump drive circuit 45 controls the operation of the above-described pressurizing pump 41 based on a control signal provided from CPU 30. Exhaust valve drive circuit 46 controls opening and closing of above-described exhaust valve 42 based on a control signal provided from CPU 30. Oscillation circuit 47 generates a signal of an oscillating frequency according to a signal provided from the pressure sensor, and outputs the generated signal toward a CPU 130. Communication unit 32 converts a signal provided from CPU 30 into electromagnetic wave of a radio frequency, transmits it to a communication unit 132 provided in display unit 100 as described later, receives electromagnetic wave transmitted from communication unit 132, converts it into an electrical signal, and inputs the signal to CPU 30. CPU 30 also serves as a blood pressure value calculation unit, and calculates a blood pressure value such as systolic pressure and diastolic pressure based on a signal provided from oscillation circuit 47. CPU 30, pressurizing pump drive circuit 45, exhaust valve drive circuit 46, oscillation circuit 47, and communication unit 32 are each configured on circuit board 38 or packaged into an IC (Integrated Circuit) to be mounted on circuit board 38.

Meanwhile, as shown in FIG. 2, display unit 100 includes CPU 130, display portion 160, a memory portion 170, a battery 180, operation portion 190, and communication unit 132. CPU 130 is a means for controlling the whole of blood pressure monitor 1 together with CPU 30 provided in cuff unit 10 as described above. Display portion 160 is formed for example of LCD (Liquid Crystal Display), and is a means for displaying the results of measurement and the like. Memory portion 170 is formed for example of ROM (Read-Only Memory) or RAM (Random-Access Memory), and is a means for storing a program to cause CPU 30, 130 and the like to execute a process procedure for measurement of the blood pressure value, and storing the results of measurement and the like. Operation portion 190 is a means for accepting operation by a subject for example and providing the externally given instruction to CPU 30, 130 or batteries 50, 180. Battery 50 is a means for supplying electric power as a power source to CPU 130. Communication unit 132 converts a signal provided from CPU 130 into electromagnetic wave of a radio frequency, transmits the wave to communication unit 32 disposed in above-described cuff unit 10, receives electromagnetic wave transmitted from communication unit 32, converts the wave into an electrical signal, and provides the signal to CPU 130. CPU 130 also provides a blood pressure value obtained as a result of measurement to memory portion 170 and display portion 160.

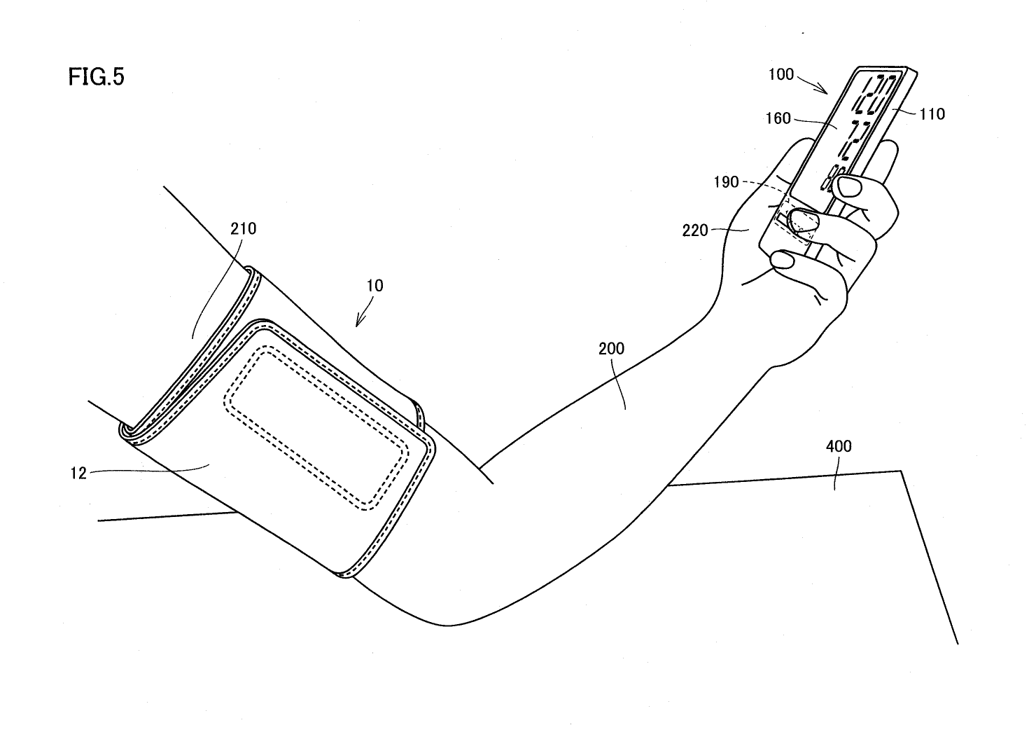

FIG. 5 is a diagram showing a state of use of the blood pressure monitor in the present embodiment. Referring now to FIG. 5, a measurement posture to be taken by a subject when using blood pressure monitor 1 in the present embodiment will be described.

As shown in FIG. 5, in using blood pressure monitor 1 in the present embodiment, the subject with cuff unit 10 wrapped around an upper arm 210 of left arm 200 positions the elbow of left arm 200 on a mount surface 400 such as table. Then, the subject holds display unit 100 with left hand 220 which is the hand of left arm 200 around which cuff unit 10 is wrapped, so that the subject can see display portion 160. When measurement is to be started, the subject operates operation portion 190 provided on display unit 100 with the thumb for example of left hand 220.

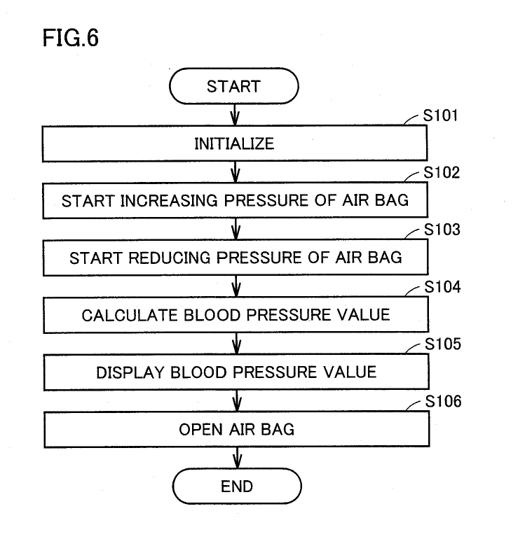

FIG. 6 is a flowchart showing a flow of a process of measuring a blood pressure value with the blood pressure monitor in the present embodiment. Referring now to FIG. 6, the flow of the process of measuring a blood pressure value with the blood pressure monitor in the present embodiment will be described. A program following this flowchart is stored in advance in memory portion 170, and CPU 30, 130 read and execute the program from memory portion 170 so that the process is executed.

When the blood pressure value is to be measured, the subject first takes the posture for measurement as shown in FIG. 5. Regarding the measurement posture shown in FIG. 5, display unit 100 is held in left hand 220. Display unit 100 may be held in the right hand instead of the left hand, or operated on a table or the like without being held in a hand. In this state, the subject operates an operation button of operation portion 190 in display unit 100 to turn on the power. Then, electric power is supplied from battery 180 as a power source to CPU 130. Subsequently, CPU 130 gives a drive instruction to CPU 30 through communication units 132, 32 and accordingly electric power is supplied from battery 50 as a power source to CPU 30. CPU 30 is thus driven and blood pressure monitor 1 is initialized as shown in FIG. 6 (step S101).

Next, CPU 30 waits for a subject's instruction to start measurement. When the subject gives the instruction to start measurement by operating operation portion 190, CPU 30 causes exhaust valve 42 to be closed and starts driving pressurizing pump 41, to thereby gradually increase the cuff pressure of air bag 20 (step S102). In the course where the pressure of air bag 20 is gradually increased, when the cuff pressure reaches a predetermined level for measuring the blood pressure value, CPU 30 stops pressurizing pump 41 and gradually opens exhaust valve 42 having been closed, to thereby gradually release the air from air bag 20 and gradually reduce the cuff pressure (step S103). Blood pressure monitor 1 in the present embodiment measures the blood pressure value in this course in which the cuff pressure is gradually reduced at an ultra-slow rate.

Next, CPU 30 calculates blood pressure values such as systolic pressure and diastolic pressure values following a known procedure (step S104). Specifically, in the course in which the cuff pressure of air bag 20 is gradually reduced, CPU 30 extracts information about pulse wave based on the oscillating frequency obtained from oscillation circuit 47. Then, from the extracted information about pulse wave, the blood pressure value is calculated. The blood pressure value is calculated in step S104, and then CPU 30 provides the blood pressure value as a result of measurement to CPU 130 through communication units 32, 132. Based on this, CPU 130 causes the blood pressure value to be displayed on display portion 160 (step S105).

After this, CPU 30 opens air bag 20 to completely release the air in air bag 20 (step S106), waits for a subject's instruction to turn off the power, and ends the operation. The measurement method described above is based on the so-called reduced-pressure measurement method by which the pulse wave is detected while the pressure of air bag 20 is reduced. It is apparently seen that the so-called increased-pressure measurement method by which the pulse wave is detected while the pressure of air bag 20 is increased may also be employed.

In blood pressure monitor 1 of the present embodiment as described above, pressurizing pump 41 and exhaust valve 42 that serve as the inflation and deflation mechanism are housed inside exterior cover 12. As for pressurizing pump 41 and exhaust valve 42, these components have been downsized to a remarkable extent recently, reduction in thickness of the profile of the components has become possible, and therefore they can be housed inside exterior cover 12 like blood pressure monitor 1 in the present embodiment. Thus, even if the structure like the present embodiment is employed, cuff unit 10 is not extremely thick, and the sufficiently good handling is maintained. When the above-described structure is employed in which the air tube is not drawn out from cuff unit 10, the blood pressure monitor excellent in handling and portability as well as storage, and the cuff for the blood pressure monitor to be equipped with the cuff can be provided. Further, because the air tube is not drawn out from cuff unit 10, handling is improved particularly of a blood pressure monitor intended to be used for a long time such as the night blood pressure monitor for measuring the blood pressure value while the subject is sleeping. The above-described increased-pressure measurement method detects the pulse wave while air is gradually fed into air bag 20, and therefore needs a smaller air capacity than the reduced-pressure measurement method. In the case where the increased-pressure measurement method is used for the above-described structure, cuff unit 20 can further be downsized and thinned.

Further, in blood pressure monitor 1 of the present embodiment, circuit board 38 on which pressure sensor 43 is mounted as well as battery 50 to serve as a power supply are also housed inside exterior cover 12. These components can also be downsized and thinned, and cuff unit 10 in which they are housed also keeps excellent handling. Thus, in the case where the blood pressure monitor is configured in this way, the associated unit configured separately from cuff unit 10 can be compact and lightweight display unit 100 provided with display portion 160 and operation portion 190 for example. Thus, the measurement can be taken while display unit 100 is held as shown in FIG. 5, display unit 100 can be operated at the position where the subject can easily manipulate it, and handling is remarkably improved. Further, in blood pressure monitor 1 of the present embodiment, cuff unit 10 and display unit 100 are configured to communicate by radio, and therefore, cuff unit 10 and display unit 100 are not connected by an electrical cable or the like, and handling, portability and storage are not deteriorated.

Furthermore, in blood pressure monitor 1 of the present embodiment, each of the variety of components as described above is individually secured to flexible curler 24, so that deformation of curler 24 is not easily hindered, and curler 24 is adequately fit on the upper arm while cuff unit 10 is mounted on the upper arm. Therefore, a gap between cuff unit 10 and the upper arm is prevented, and the blood pressure can be measured with high precision without deterioration in fitting. At a portion located between components arranged and secured along the circumferential direction of curler 24, a groove or a thickness-reduced portion extending in the axial direction may be provided or a rotational mechanism may be provided, so as to allow curler 24 to surely fit on the upper arm while cuff unit 10 is mounted on the upper arm. Therefore, this structure can further ensure that deterioration in fitting is prevented.

As heretofore described, when blood pressure monitor 1 and cuff unit 10 with which the blood pressure monitor is equipped are configured in the manner like the present embodiment, the blood pressure monitor that can be downsized and is excellent in handling and portability as well as the cuff for the blood pressure monitor to be equipped with the cuff can be provided.

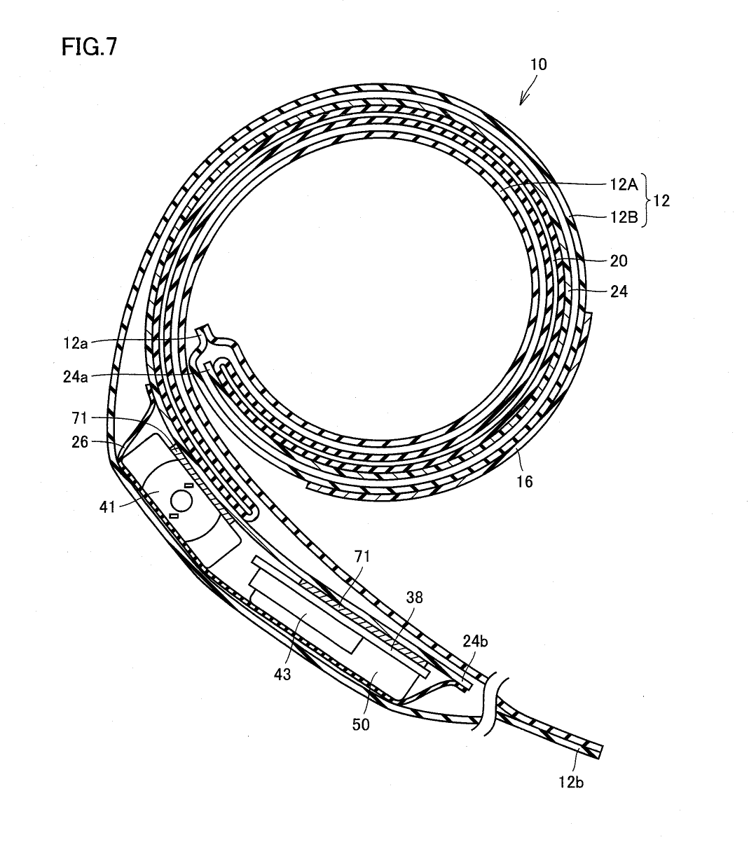

FIG. 7 is a cross section of a cuff unit showing a modification of the blood pressure monitor in the embodiment as described above. In the cuff unit with which the blood pressure monitor is equipped in the present embodiment, the various kinds of components mounted on the external peripheral surface of the curler are directly covered with the outer cover of the exterior cover. In contrast, in cuff unit 10 of the modification, as shown in FIG. 7, a resin film 26 to serve as a protection member is provided between various kinds of components including the inflation and deflation mechanism and outer cover 12B, and the exposed surface of each component is covered with resin film 26. For resin film 26, according to one or more embodiments of the present invention, a material having its surface with small friction coefficient is used, and formed of a thin film for example of polypropylene (PP), or polyethylene terephthalate (PET) or the like. According to one or more embodiments of the present invention, the opposite two ends of resin film 26 are secured to curler 24.

This structure can be used to prevent various kinds of components secured on curler 24 from deteriorating in terms of the secured state due to repeated use and resultant friction with exterior cover 12, and obtain the effect that curler 24 on which these various kinds of components are secured in assembly can be easily inserted in exterior cover 12.

### Second Embodiment

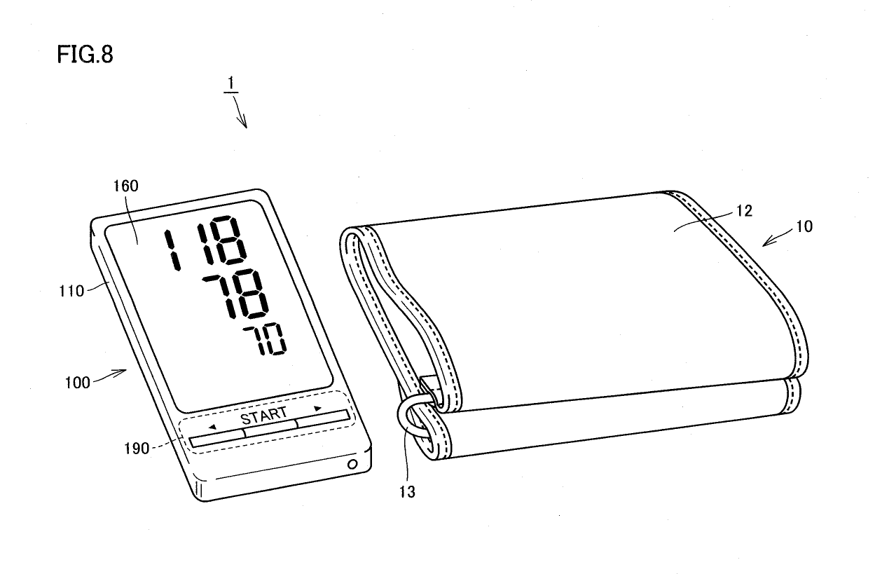

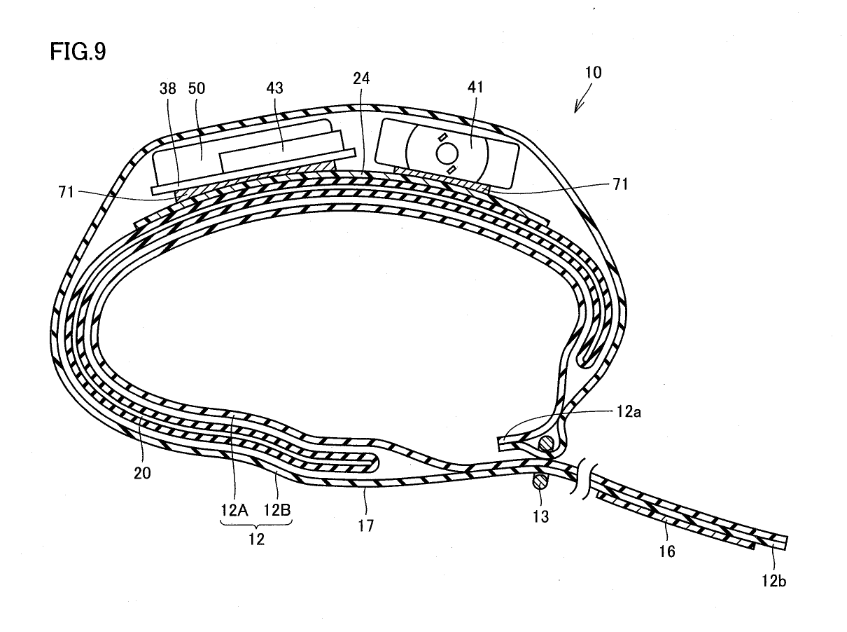

FIG. 8 is a perspective view showing an external structure of a blood pressure monitor in a second embodiment of the present invention. FIG. 9 is a schematic cross section of a cuff unit shown in FIG. 8. In the following, a description will be given with reference to FIGS. 8 and 9 of the structure of the blood pressure monitor and a cuff for the blood pressure monitor to be equipped with the cuff in the second embodiment. Those components similar to corresponding ones of the blood pressure monitor and the cuff for the blood pressure monitor to be equipped with the cuff in the first embodiments are denoted by the same reference characters in the drawings, and the description thereof will not be repeated here.

As shown in FIGS. 8 and 9, blood pressure monitor 1 in the present embodiment differs in structure from blood pressure monitor 1 and cuff unit 10 in the first embodiment. As to cuff unit 10 in the present embodiment, a ring member 13 is attached to inner end 12a of exterior cover 12, and a portion relatively closer to outer end 12b of exterior cover 12 is inserted in ring member 13. On the outer peripheral surface of a portion relatively closer to outer end 12b than the portion inserted in ring member 13 of exterior cover 12, hook-and-loop fastener part 16 is provided. On the outer peripheral surface of a portion relatively closer to inner end 12a than the portion inserted in ring member 13 of exterior cover 12, hook-and-loop fastening part 17 is provided. In cuff unit 10 of the present embodiment, a portion of exterior cover 12 located closer to outer end 12b than the portion inserted in ring member 13 is folded back at ring member 13, and hook-and-loop fastener part 16 provided on the folded-back portion of exterior cover 12 is engaged with above-described hook-and-loop fastener part 17, so that cuff unit 10 is wrapped around and secured to an upper arm.

Further, in cuff unit 10 of the present embodiment, curler 24 is formed in the shape of an arc. Specifically, while the curler of the cuff unit in the first embodiment is formed to have an annular shape, curler 24 in cuff unit 10 of the present embodiment has a considerably reduced circumferential length to leave a length necessary for various kinds of components such as inflation and deflation mechanism to be secured to curler 24 to be mounted. As seen from the above, while curler 24 of the cuff unit in the first embodiment is annular in shape so that the tubular form is kept, the annular form of curler 24 of cuff unit 10 in the present embodiment is not kept by curler 24.

The above-described structure can also provide effects similar to the effects explained in connection with the first embodiment above. Specifically, the air tube is not drawn out from cuff unit 10, and the associated unit formed separately from cuff unit 10 can be implemented as compact and lightweight display unit 100 provided with display portion 160 and operation portion 190 for example. Thus, the blood pressure monitor that can be downsized and is excellent in handling and portability as well as the cuff for the blood pressure monitor to be equipped with the cuff can be provided.

### Third Embodiment

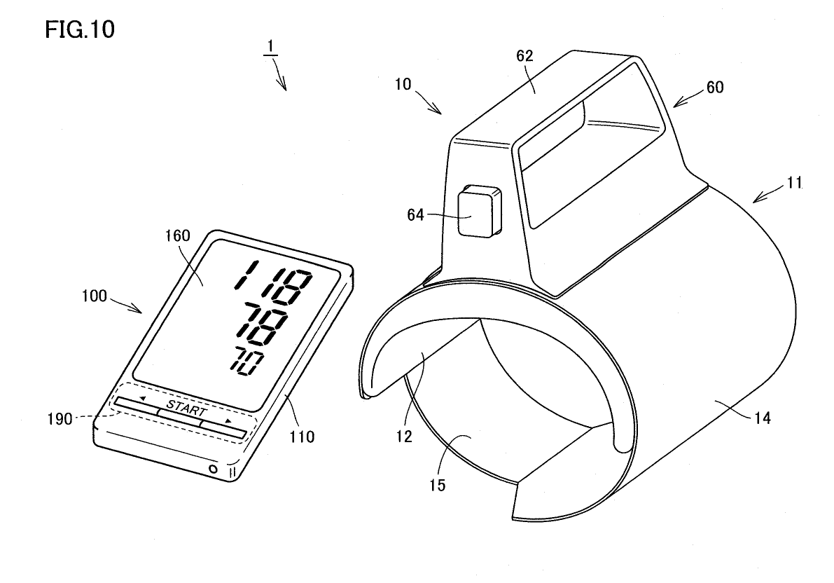

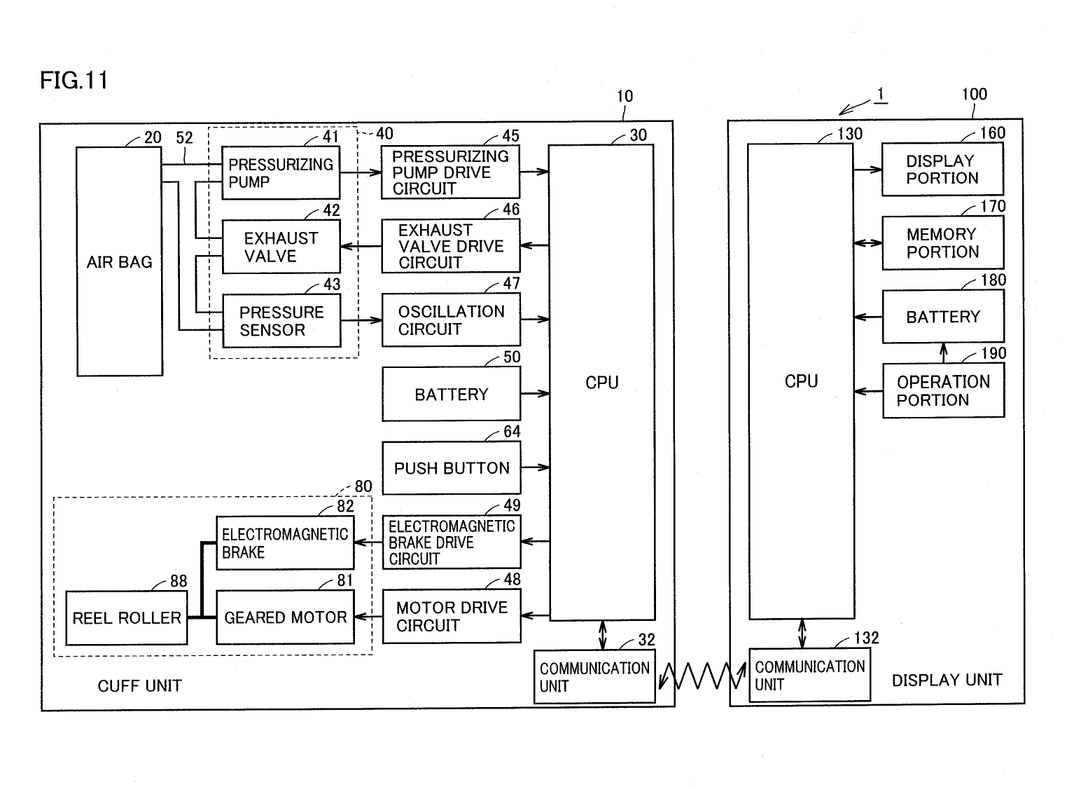

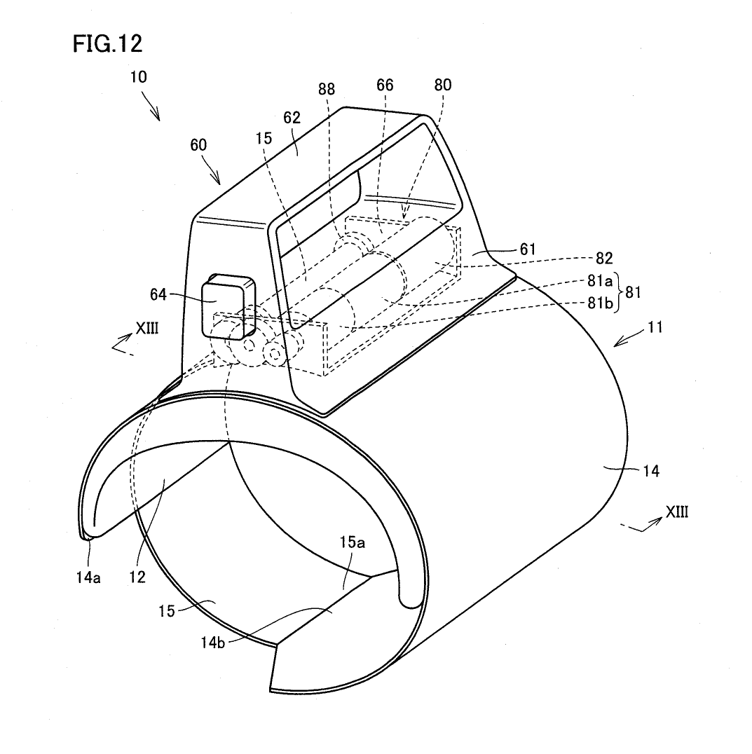

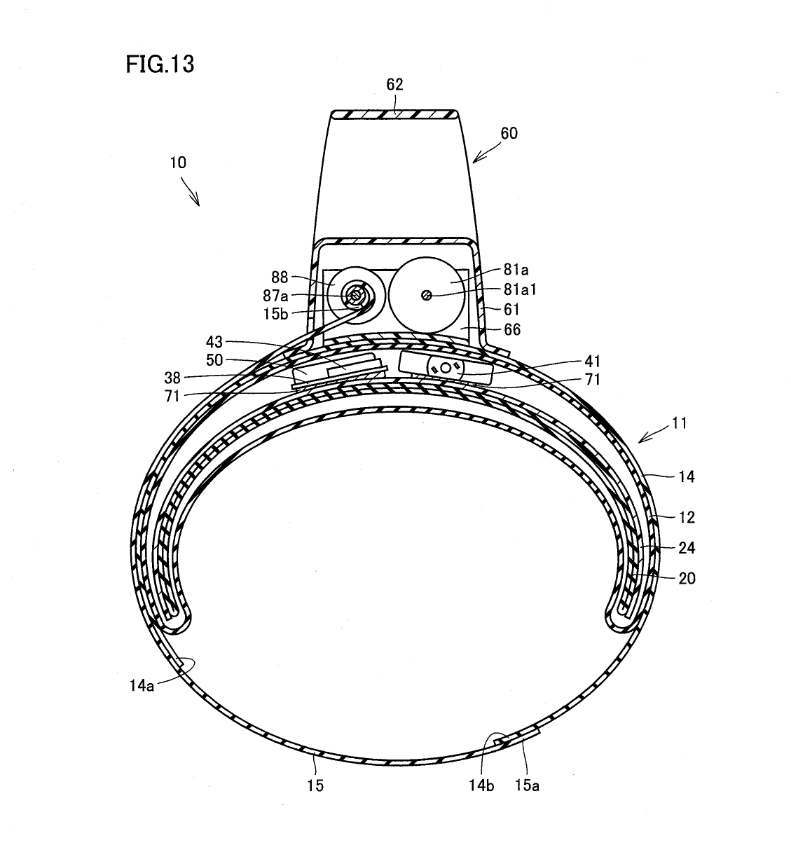

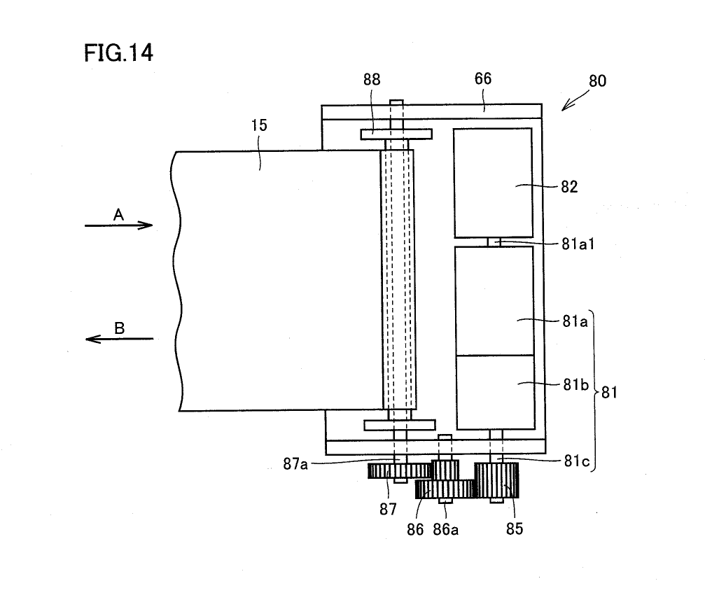

FIG. 10 is a perspective view showing an external structure of a blood pressure monitor in a third embodiment of the present invention. FIG. 11 is a block diagram showing a configuration of functional blocks of the blood pressure monitor shown in FIG. 10. FIG. 12 is a perspective view showing a detailed structure of a cuff unit shown in FIG. 10. FIG. 13 is a schematic cross section of a cuff along line XIII-XIII shown in FIG. 12. FIG. 14 is a schematic top view showing a structure of a tightening length adjustment mechanism shown in FIG. 12. Referring first to FIGS. 10 to 14, the structure of the blood pressure monitor and the cuff for the blood pressure monitor to be equipped with the cuff in the third embodiment of the present invention will be described. Those components similar to corresponding ones of the blood pressure monitor and the cuff for the blood pressure monitor to be equipped with the cuff in the first embodiment are denoted by the same reference characters in the drawings, and the description thereof will not be repeated here.

The blood pressure monitor in the present embodiment is configured so that a tightening operation of the cuff unit on an upper arm, an operation of measuring the blood pressure value that is performed after the tightening operation, and a tightening release operation of the cuff unit on the upper arm that is performed after the measuring operation are automatically carried out successively.

As shown in FIGS. 10 to 13, blood pressure monitor 1 in the present embodiment differs in structure from blood pressure monitor 1 and cuff unit 10 in the above-described first embodiment. Cuff unit 10 in the present embodiment includes a tubular cuff body 11 to be mounted on an upper arm, and a grip portion 60 provided on the outer peripheral surface of cuff body 11. Grip portion 60 includes a base 61 attached to cuff body 11, and a grip 62 to be gripped in a hand when the cuff unit is to be mounted. Cuff body 11 is tubular-shaped to allow the upper arm to be inserted along the axial direction. Grip portion 60 is secured to cuff body 11 so that grip 62 extends in parallel with the axial direction of tubular-shaped cuff body 11. A tightening length adjustment mechanism 80 described later is located on the outer peripheral surface of cuff body 11 and inside base 61 of grip portion 60. At a predetermined position of grip portion 60, a push button 64 is provided.

As shown in FIGS. 10 and 12, cuff body 11 mainly includes annularly extended tightening belts 14 and 15, exterior cover 12 attached to the inside of tightening belt 14, and curler 24 and air bag 20 that are housed inside exterior cover 12. Tightening belts 14, 15 are each formed of a member such as cloth substantially having no circumferential stretchability, and include a first tightening belt 14 of a relatively large width, and a second tightening belt 15 of a relatively small width coupled to first tightening belt 14.

First tightening belt 14 is formed of a belt-like member having one end 14a and the other end 14b with respect to the circumferential direction, above-described grip portion 60 is attached at a predetermined position to the outer peripheral surface of the first tightening belt, and above-described exterior cover 12 is attached onto the inner peripheral surface thereof. Second tightening belt 15 has one end 15a and the other end 15b with respect to the circumferential direction, and one end 15a is coupled to the other end 14b of first tightening belt 14. Second tightening belt 15 has a portion which is located relatively closer to the other end 15b and placed on the outer periphery of a portion relatively closer to one end 14a of first tightening belt 14, and the other end 15b of second tightening belt 15 is secured to a reel roller 88 disposed inside grip portion 60 attached onto the outer peripheral surface of first tightening belt 14. Accordingly, first tightening belt 14 and second tightening belt 15 function as a united and annularly-configured member and thereby form cuff body 11 having a hollow opening.

Coupled first tightening belt 14 and second tightening belt 15 have the circumferential length that is variably adjusted by tightening length adjustment mechanism 80 described later. While the circumferential length of coupled first tightening belt 14 and second tightening belt 15 is adjusted to be long, cuff body 11 has an increased-diameter state (the state where the diameter is increased). While the circumferential length thereof is adjusted to be short, cuff body 11 has a reduced-diameter state (the state where the diameter is reduced).

Exterior cover 12 is formed of a member such as cloth made of a stretchable and low-friction material for example, and attached onto the inner peripheral surface of above-described first tightening belt 14. More specifically, the outer peripheral surface of exterior cover 12 is joined by bonding, welding or the like to the inner peripheral surface of first tightening belt 14, and accordingly exterior cover 12 is secured to first tightening belt 14.

Curler 24 encapsulated in exterior cover 12 is arc-shaped and configured to be elastically deformable in the radial direction. Here, as shown in FIG. 13, in cuff unit 10 of the present embodiment as well, circuit board 38 on which pressurizing pump 41 and exhaust valve 42 (see FIG. 11), an air joint (not shown), and pressure sensor 43 are mounted, as well as battery 50 for example are disposed on the outer peripheral surface of curler 24. Namely, these different kinds of components are housed in the space inside exterior cover 12.

Curler 24 in the above-described diameter-increased state assumes a state in which the diameter of the curler is increased to be larger. On the contrary, curler 24 in the above-described diameter-reduced state assumes a state in which the diameter of the curler is decreased to be smaller. When cuff body 11 assumes the diameter-increased state, the elastic force of curler 24 causes cuff body 11 to be enlarged, which facilitates insertion and removal of an upper arm into and from the hollow opening of cuff body 11.

As shown in FIG. 11, cuff unit 10 includes, in addition to the functional blocks included in the cuff unit of the above-described first embodiment, push button 64, a geared motor 81, an electromagnetic brake 82, reel roller 88, a motor drive circuit 48, and an electromagnetic brake drive circuit 49. Geared motor 81, electromagnetic brake 82, and reel roller 88 correspond to tightening length adjustment mechanism 80 that variably adjusts the tightening length relative to an upper arm of tightening belts 14, 15 as described above. In blood pressure monitor 1 of the present embodiment, air bag 20 and air system component 40 are used as a tightening force detection mechanism for detecting the tightening force of tightening belts 14, 15 exerted on the upper arm, and details of this will be described later.

Geared motor 81 is an electric motor rotationally driving reel roller 88 in the forward and reverse directions, and the operation of the motor is controlled by motor drive circuit 48. Electromagnetic brake 82 is a brake applying a braking force to reel roller 88, and the operation of the brake is controlled by electromagnetic brake drive circuit 49. Reel roller 88 is a member for reeling and feeding above-described tightening belts 14, 15. Push button 64 is a portion receiving a subject's instruction to start the tightening operation of tightening length adjustment mechanism 80 constituted of geared motor 81, electromagnetic brake 82, and reel roller 88.

Motor drive circuit 48 controls the operation of geared motor 81 based on a control signal that is input from CPU 30. Electromagnetic brake drive circuit 49 controls the operation of electromagnetic brake 82 based on a control signal that is input from CPU 30. In blood pressure monitor 1 of the present embodiment, CPU 30 provided in cuff unit 10 controls driving of pressurizing pump 41 and exhaust valve 42, and further controls driving of above-described geared motor 81 and electromagnetic brake 82, and battery 50 provided in cuff unit 10 supplies electric power for driving to geared motor 81 and electromagnetic brake 82.

As shown in FIGS. 12 to 14, tightening length adjustment mechanism 80 includes geared motor 81, electromagnetic brake 82, and reel roller 88. These geared motor 81, electromagnetic brake 82, and reel roller 88 are provided on a support frame 66 disposed at a position on the outer peripheral surface of cuff body 11 and inside base 61 of grip portion 60. Support frame 66 is secured for example onto the outer peripheral surface of first tightening belt 14. At respective predetermined positions of support frame 66, gears 85, 86, 87 to serve as a power transmission are provided on the support frame.

Geared motor 81 is a motor equipped with a reducer, and includes a motor portion 81a, a reducer portion 81b, and an output shaft 81c. To output shaft 81c of geared motor 81, gear 85 is fixed. At the axial end opposite to the side where output shaft 81c of geared motor 81 is located, electromagnetic brake 82 is disposed adjacent to this geared motor 81. Electromagnetic brake 82 exerts a braking force on a rotational shaft 81a1 by binding rotational shaft 81a1 of motor portion 81a.

Reel roller 88 is fixed to a shaft 87a which is axially supported on support frame 66, and rotated to follow the rotation of shaft 87a. To reel roller 88, the other end 15b of second tightening belt 15 is secured as described above. To shaft 87a to which reel roller 88 is fixed, gear 87 is fixed. To a shaft 86a axially supported on support frame 66, gear 86 is fixed. Gear 86 meshes with these gears 85 and 87 each to transmit the rotational force generated on output shaft 81c of geared motor 81 to reel roller 88. These gears 85, 86, 87 are configured with respective outer diameters and respective numbers of teeth that are adjusted, and also function as a reducer like reducer portion 81b of geared motor 81.

Referring next to FIG. 14, the operation of tightening length adjustment mechanism 80 provided in cuff unit 10 of the present embodiment will be described. In blood pressure monitor 1 of the present embodiment, as described above, the tightening operation of cuff unit 10 on an upper arm, the operation of measuring the blood pressure value that is performed after the tightening operation, and the tightening release operation of cuff unit 10 on the upper arm that is performed after the measuring operation are automatically carried out successively. Of these operations, the tightening operation of cuff unit 10 on the upper arm and the tightening release operation of cuff unit 10 on the upper arm are effected respectively through a reeling operation of tightening belts 14, 15 caused by tightening length adjustment mechanism 80, and a feeding operation of tightening belts 14, 15 caused by tightening length adjustment mechanism 80 that are each described in the following.

With reference to FIG. 14, when geared motor 81 is driven to rotate in the forward direction, output shaft 81c of geared motor 81 rotates in the forward direction, the rotational force is transmitted through gears 85, 86, 87 to shaft 87a, and reel roller 88 rotates in the forward direction. The forward rotation of reel roller 88 causes second tightening belt 15 having its other end 15b secured to reel roller 88 to be reeled up by reel roller 88 in the direction indicated by an arrow A in FIG. 14. The reeling operation of second tightening belt 14 effected by reel roller 88 causes the tightening length of tightening belts 14, 15 to be reduced against the elastic force of curler 24, and the diameter of the hollow opening of cuff body 11 is gradually reduced. Namely, the reeling operation implements the tightening operation of cuff unit 10 on an upper arm. While geared motor 81 is driven to rotate in the forward direction, electromagnetic brake 82 does not bind rotational shaft 81a1 of motor portion 81a of geared motor 81, and motor portion 81a is driven without restricted in its operation.

In contrast, when geared motor 81 is driven to rotate in the reverse direction, output shaft 81c of geared motor 81 rotates in the reverse direction, the rotational force is transmitted through gears 85, 86, 87 to shaft 87a, and reel roller 88 rotates in the reverse direction. The rotation in the reverse direction of reel roller 88 causes second tightening belt 15 having been reeled around reel roller 88 to be fed from reel roller 88 in the direction indicated by an arrow B in FIG. 14. The feeding operation of second tightening belt 15 effected by reel roller 88 increases the tightening length of tightening belts 14, 15. At this time, the diameter of the hollow opening of cuff body 11 is gradually increased based on the elastic force of curler 24. Namely, the feeding operation implements the tightening release operation of cuff unit 10 on the upper an n. While geared motor 81 is driven to rotate in the reverse direction, electromagnetic brake 82 does not bind rotational shaft 81a1 of motor portion 81a of geared motor 81, and motor portion 81a is driven without being restricted in its operation.

While geared motor 81 is not driven to rotate in the forward and reverse directions, namely geared motor 81 is stopped, rotational shaft 81a1 of motor portion 81a of geared motor 81 is bound by electromagnetic brake 82. In this state, the braking force of electromagnetic brake 82 is exerted on reel roller 88 through rotational shaft 81a1 of motor portion 81a, reducer portion 81b, output shaft 81c, gears 85, 86, 87, and shaft 87a, and thereby restricts the rotational operation of reel roller 88. Therefore, in this state, both of the reeling and feeding operations of second tightening belt 15 effected by reel roller 88 are stopped, and the diameter of the hollow opening of cuff body 11 is kept constant.

Next, the tightening force detection mechanism provided to blood pressure monitor 1 in the present embodiment will be described. The tightening force detection mechanism serves to detect the tightening force of cuff unit 10 on an upper arm during the tightening operation of cuff unit 10 as described above, for the purpose of making optimum the tightening state of cuff unit 10 on the upper arm.

As seen from the description above, in blood pressure monitor 1 of the present embodiment, the tightening force detection mechanism is constituted of air bag 20 and air system component 40. The tightening force detection mechanism serves to detect the tightening force of tightening belts 14, 15 on an upper arm, and acquires the tightening force as the internal pressure of air bag 20.

Specifically, in blood pressure monitor 1 of the present embodiment, before tightening length adjustment mechanism 80 is driven to reduce the diameter of the hollow opening of cuff body 11, a predetermined amount of air is injected by pressurizing pump 41 into air bag 20. As above-described tightening length adjustment mechanism 80 is driven to reduce the tightening length, the internal pressure of air bag 20 held between tightening belts 14, 15 and the upper arm is detected with pressure sensor 43, and accordingly the tightening force exerted by tightening belts 14, 15 on the upper arm is detected based on the detected internal pressure of air bag 20.

CPU 30 monitors the internal pressure of above-described air bag 20 while tightening length adjustment mechanism 80 is performing the tightening operation, stops the operation of geared motor 81 when the internal pressure reaches a predetermined pressure value, and simultaneously operates electromagnetic brake 82 to stop the rotation of reel roller 88. In this way, the tightening state of cuff unit 10 on the upper arm can be made optimum.

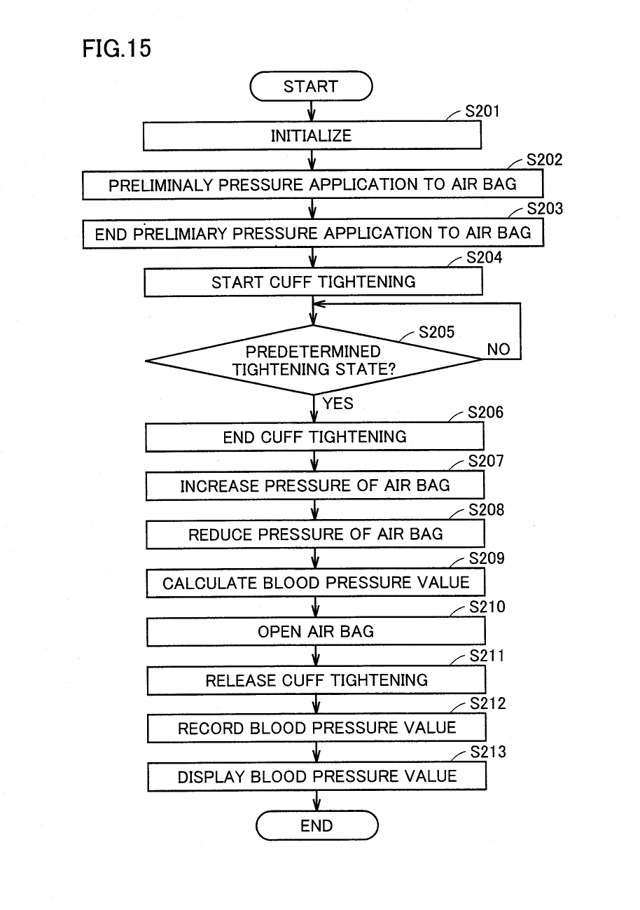

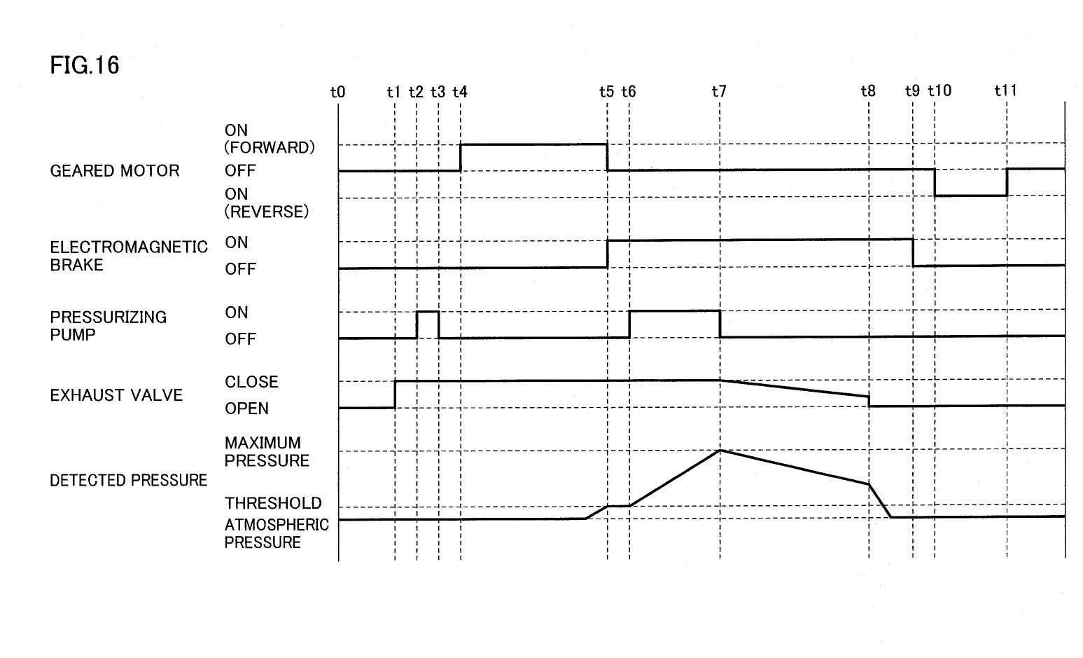

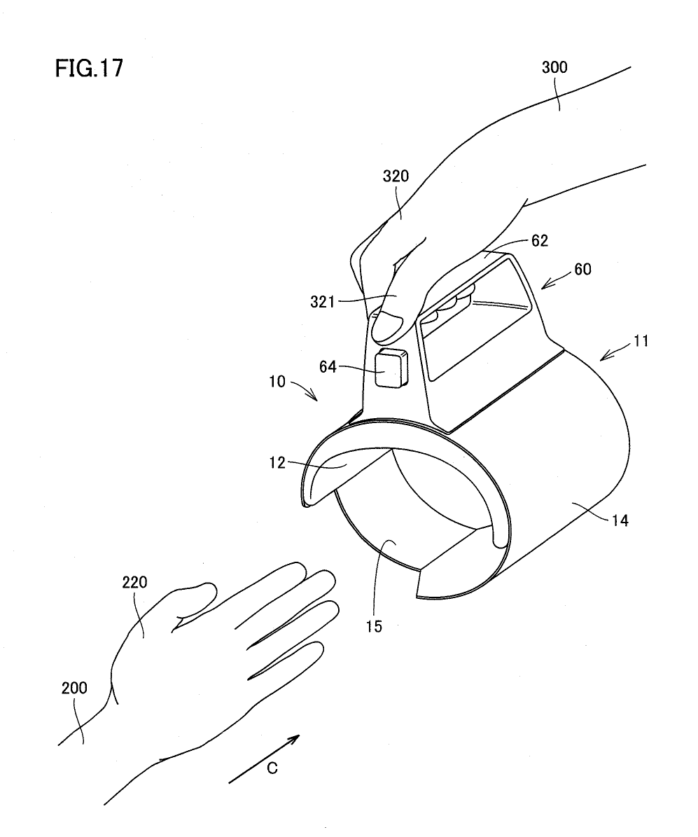

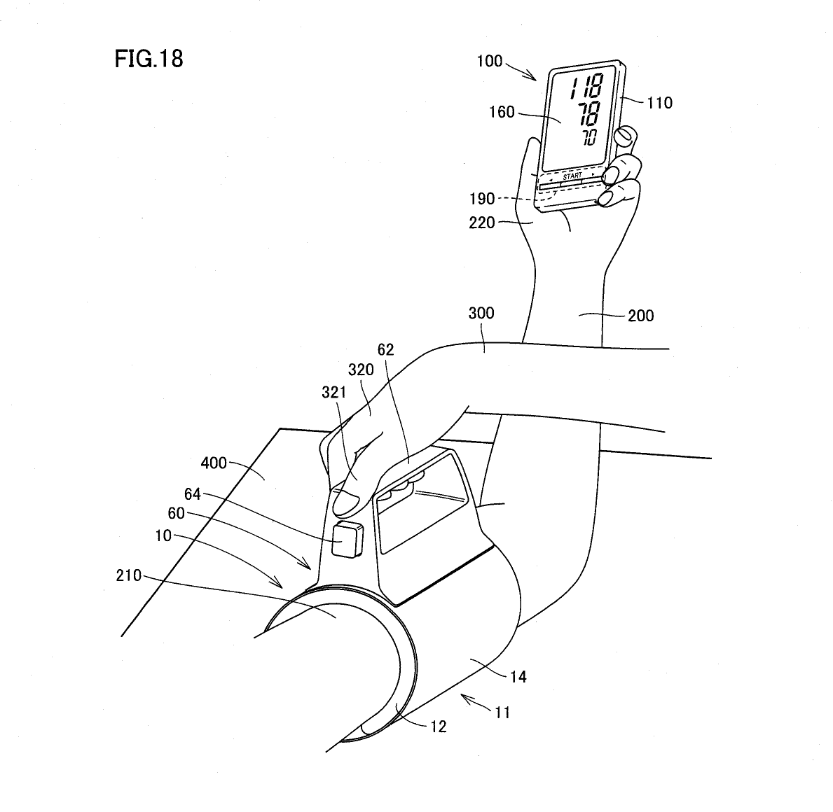

FIG. 15 is a flowchart showing a process procedure of the blood pressure monitor in the present embodiment. FIG. 16 is a timing chart showing in time series manner an operational condition or operational state of each component of the blood pressure monitor in the present embodiment. FIG. 17 is a schematic diagram for illustrating a mount operation of mounting the cuff unit in the present embodiment on an upper arm. FIG. 18 is a schematic diagram of a mount state where the cuff unit in the present embodiment is mounted on an upper arm. Referring now to FIGS. 15 to 18, a process procedure of blood pressure monitor 1 in the present embodiment will be described together with the operational condition or operational state of each component of blood pressure monitor 1, or the mount operation for cuff unit 10 and the state after cuff unit 10 is mounted. It should be noted that the program following the flowchart shown in FIG. 15 is stored in advance in memory portion 170 shown in FIG. 11, and CPU 300 reads the program from memory portion 170 and executes the program to proceed with the process.

First, as shown in FIG. 15, a subject operates an operation button of operation portion 190 in display unit 100 so as to turn on the power. Then, battery 180 supplies electric power as a power source to CPU 130. Subsequently, CPU 130 gives a drive instruction through communication units 132, 32 to CPU 30, and accordingly electric power is supplied from battery 50 as a power source to CPU 30. Thus, CPU 30 is driven and blood pressure monitor 1 is initialized (step S201). As shown in FIG. 16, at time t0 when blood pressure monitor 1 is initialized, respective operations of geared motor 81, electromagnetic brake 82, and pressurizing pump 41 are stopped, namely in the OFF state, exhaust valve 42 is in the opened state allowing the internal space of air bag 20 to communicate with the outside and making the cuff pressure equal to the atmospheric pressure, and the cuff pressure detected by pressure sensor 43 has the same value as the atmospheric pressure.

Next, the subject holds grip 62 of cuff unit 10 in right hand 320, and inserts left hand 220, which is a different hand from right hand 320 holding grip 62, in the direction of an arrow C shown in FIG. 17 into the hollow opening of cuff body 11 of cuff unit 10. Then, as shown in FIG. 18, the subject moves cuff unit 10 to upper arm 210 of left arm 200, keeps the state where cuff unit 10 fits on upper arm 210 of left arm 200, while pressing push button 64 provided to grip 62 with thumb 321 of right hand 320 which holds grip 62.

As shown in FIG. 15, in response to pressing of push button 64 by the subject, CPU 30 causes preliminary pressure application to air bag 20 (step S202). Specifically, as shown in FIG. 16, at time t1 when push button 64 is pressed, CPU 30 closes exhaust valve 42 for inhibiting the internal space of air bag 20 from communicating with the outside, and subsequently starts, at time t2, driving pressurizing pump 41 for injecting air into air bag 20. Then, CPU 30 stops driving pressurizing pump 41 at time t3 when a predetermined time has elapsed. Here, the time for which pressurizing pump 41 is driven is a time necessary for allowing a predetermined amount of air to be injected into the internal space of air bag 20. Accordingly, the preliminary pressure application to air bag 20 is completed (step S203).

Next, as shown in FIG. 15, CPU 30 starts the tightening operation of cuff unit 10 on the upper arm (step S204). At this time, CPU 30 determines whether or not the tightening of cuff unit 10 on the upper arm has reached a predetermined tightening state (step S205). When the predetermined tightening state has not been reached (NO in step S205), CPU 30 continues the tightening operation of cuff unit 10 on the upper arm. When the predetermined tightening state has been reached (YES in step S205), CPU 30 stops the tightening operation of cuff unit 10 on the upper arm (step S206).

Specifically, as shown in FIG. 16, at time t4, CPU 30 drives geared motor 81 to rotate in the forward direction to thereby start the reeling operation of second tightening belt 15 effected by reel roller 88. Then, CPU 30 causes pressure sensor 43 to detect the cuff pressure of air bag 20 while the reeling operation is performed. At time t5 when the detected cuff pressure reaches a predetermined threshold, CPU 30 stops driving for causing the rotation in the forward direction of geared motor 81, and simultaneously drives electromagnetic brake 82 to stop the rotation of reel roller 88. Here, the above-described threshold is determined in advance based on a tightening force of cuff unit 10 on the upper arm that is suitable for measuring the blood pressure value.

Here, while cuff unit 10 is tightened with an optimum tightening force on upper arm 210, it is ensured that tightening belts 14, 15 press air bag 20 against upper arm 210. Therefore, in the subsequent measuring operation, inflation of air bag 20 ensures that upper arm 210 is compressed by cuff unit 10, and accordingly ensures that the blood flow in the artery located inside upper arm 210 is interrupted.

Next, as shown in FIG. 15, CPU 30 starts increasing the pressure of air bag 20 for the purpose of measuring the blood pressure value (step S207). Specifically, as shown in FIG. 16, CPU 30 drives at time t6 pressurizing pump 41 to increase the cuff pressure, so that the pressure of air bag 20 is increased to reach a predetermined cuff pressure.

Next, as shown in FIG. 15, CPU 30 starts an ultra-slow rate pressure reduction of air bag 20 for the purpose of measuring the blood pressure value (step S208). Specifically, as shown in FIG. 16, CPU 30 stops driving pressurizing pump 41 at time t7 when pressure sensor 43 detects that the internal pressure of air bag 20 reaches a predetermined internal pressure, and subsequently controls the extent to which exhaust valve 42 is opened while gradually opening exhaust valve 42. At this time, CPU 30 acquires a variation of the cuff pressure detected by pressure sensor 43.

Next, as shown in FIG. 15, CPU 30 calculates the blood pressure value based on the variation of the cuff pressure acquired in the ultra-slow rate pressure reduction process (step S209). Subsequently, CPU 30 opens air bag 20 (step S210) and releases tightening of cuff unit 10 on upper arm 210 (step S211). Specifically, as shown in FIG. 16, at time t8 when calculation of the blood pressure value is completed, CPU 30 completely opens exhaust valve 42 to release the air in air bag 20 to the outside. Subsequently, at time t9, CPU 30 stops the operation of electromagnetic brake 82. At time t10, CPU 30 drives geared motor 81 to rotate in the reverse direction and thereby cause second tightening belt 15 to be fed from reel roller 88. After this, at time t11 when second tightening 15 is completely fed from reel roller 88, CPU 30 stops driving geared motor 81. Here, stoppage of driving of geared motor 81 is controlled based on time. Instead, control may be performed in such a manner that detection means such as optical sensor is provided to cuff body 11 to detect the fact that the endmost portion of second tightening belt 15 is fed out from reel roller 88 and then stop driving of geared motor 81.

Next, as shown in FIG. 15, CPU 30 inputs the blood pressure value obtained from the measurement to CPU 130 through communication units 32, 132. Based on this, CPU 130 outputs the blood pressure value to memory portion 170 and display portion 160. Memory portion 170 stores the blood pressure value obtained as a result of the measurement (step S212), and display portion 160 displays the blood pressure value as the result of the measurement (step S213). Here, display portion 160 displays the systolic pressure value and the diastolic pressure value in numerical values for example. After these blood pressure values are recorded and displayed, blood pressure monitor 1 enters a standby mode and, after a subject's instruction to turn off the power is given to operation portion 190, stops supply of electric power as a power source.

Regarding blood pressure monitor 1 configured as described above, a very simple operation of holding grip 62 provided to cuff unit 10 in right hand 320 of the arm which is different from upper arm 210 of left arm 200 on which cuff unit 10 is to be mounted, inserting left hand 220 of the arm, on which cuff unit 10 is to be placed, in the hollow opening of cuff unit 10 in this state and fitting cuff unit 10 on upper arm 210, and thereafter pressing push button 64 provided to grip 62 is performed, so that the tightening operation of cuff unit 10 on upper arm 210 is automatically performed subsequently. Therefore, cuff unit 10 can be mounted very easily on upper arm 210 which is a site for measurement.

Further, blood pressure monitor 1 in the present embodiment as described above is configured to detect, when tightening belts 14, 15 are used to tighten upper arm 210, the tightening force of cuff unit 10 on upper arm 210 with the tightening force detection mechanism, and keep the state where the tightening force is optimized by means of tightening length adjustment mechanism 80. Therefore, each time the measurement is to be taken, cuff unit 10 is reliably wrapped around upper arm 210.

Accordingly, the above-described configuration can be employed to facilitate mounting of cuff unit 10 on upper aim 210, and enable cuff unit 10 to be reliably wrapped around upper arm 210 each time the measurement is to be taken. As a result, the blood pressure monitor capable of precisely and stably measuring the blood pressure value and is excellent in convenience for use can be provided.

Blood pressure monitor 1 in the present embodiment is configured in such a manner that not only mounting of above-described cuff unit 10, but also the subsequently performed operation of measuring the blood pressure value, and the operation of releasing tightening of cuff unit 10 on upper arm 210 after the measuring operation are all automatically carried out successively. Therefore, such a configuration can be employed to provide the blood pressure monitor which enables cuff unit 10 to be mounted, the blood pressure value to be measured, and cuff unit 10 to be removed by a so-called one-touch operation and which is accordingly very excellent in convenience for use.

Further, blood pressure monitor 1 in the present embodiment is configured in such a manner that geared motor 81, electromagnetic brake 82, and reel motor 88 that serve as tightening length adjustment mechanism 80 are housed in base 61 of grip portion 60. Therefore, cuff unit 10 can be small-sized and compact, and an effect that increase in size of cuff unit 10 is avoided can also be obtained.

In addition, from blood pressure monitor 1 in the present embodiment, similar effects to those described in connection with the first embodiment above can be obtained. Specifically, the air tube is not drawn out from cuff unit 10, and the associated unit configured separately from cuff unit 10 can be provided as compact and lightweight display unit 100 including display portion 160 and operation portion 190 for example. Thus, the blood pressure monitor that can be downsized and is excellent in handling and portability as well as the cuff for the blood pressure monitor to be equipped with the cuff can be provided.

Blood pressure monitor 1 in the present embodiment has been described in connection with an example where air bag 20 and air system component 40 are used as the tightening force detection mechanism. Instead, a torque sensor for detecting the rotational torque applied to reel roller 88 on which second tightening belt 15 is wound may be provided, and the torque sensor may be used to detect the tightening force of tightening belts 14, 15 on upper arm 210 as the rotational torque applied to reel roller 88.

Blood pressure monitor 1 in the present embodiment has also been described in connection with an example where push button 64 constitutes an operation portion for starting the reeling operation of second tightening belt 15. The operation portion, however, is not necessarily formed of the push button and, a slide-type button, dial-type button, touch sensor, a voice recognition sensor, or the like may also be used to constitute the operation portion.

The first to third embodiments of the present invention have been described in connection with an example where the site on which the blood pressure monitor is mounted is the left upper arm. It is apparently seen that the site on which the blood pressure monitor is mounted may be the right upper arm. The first to third embodiments have also been described in connection with an example where the blood pressure monitor is a so-called upper-arm blood pressure monitor having a cuff unit mounted on an upper arm for measuring the blood pressure value. The blood pressure monitor, however, is not particularly limited to this. One or more embodiments of the present invention is also applicable to a so-called wrist blood pressure monitor having a cuff unit mounted on a wrist for measuring the blood pressure value, a so-called ankle blood pressure monitor having a cuff unit mounted on an ankle for measuring the blood pressure value, and the like.

Further, the first to third embodiments of the present invention have been described in connection with an example where one or more embodiments of the present invention is applied to a blood pressure monitor capable of measuring the systolic pressure and diastolic pressure values. One or more embodiments of the present invention is also applicable to a blood pressure information measuring device capable of measuring information about the blood pressure other than the systolic pressure and diastolic pressure values, such as mean blood pressure value, pulse wave, pulse rate, AI (Augumentation Index), and the like.

The embodiments and their modification disclosed herein are given by way of illustration in all respects, not by way of limitation. While the invention has been described with respect to a limited number of embodiments, those skilled in the art, having benefit of this disclosure, will appreciate that other embodiments can be devised which do not depart from the scope of the invention as disclosed herein. It is intended that the technical scope of the present invention is defined by claims, and encompasses all variations equivalent in meaning and scope to the claims.

### DESCRIPTION OF THE REFERENCE SIGNS

1 blood pressure monitor; 10 cuff unit; 11, cuff body; 12 exterior cover; 12A inner cover; 12B outer cover; 12a inner end; 12b outer end; 13 ring member; 14, 15 tightening belt; 14a, 15a one end; 14b, 15b other end; 16, 17 hook-and-loop fastener parts; 18 mark; 20 air bag; 24 curler; 24a inner end; 24b outer end; 26 resin film; 30 CPU; 32 communication unit; 38 circuit board; 40 air system component; 41 pressurizing pump; 42 exhaust valve; 43 pressure sensor; 44 air joint; 45 pressurizing pump drive circuit; 46 exhaust valve drive circuit; 47 oscillation circuit; 48 motor drive circuit; 49 electromagnetic brake drive circuit; 50 battery; 52 air tube; 60 grip portion; 61 base; 62 grip; 64 push button; 66 support frame; 71 adhesive sheet; 80 tightening length adjustment mechanism; 81 geared motor; 81a motor portion; 81a1 rotational shaft; 81b reducer portion; 81c output shaft; 82 electromagnetic brake; 85, 86, 87 gear; 86a, 87a shaft; 88 reel roller; 100 display unit; 110 casing; 130 CPU; 132 communication unit; 160 display portion; 170 memory portion; 180 battery; 190 operation portion; 200 left arm; 210 upper arm; 220 left hand; 300 right arm; 320 right hand; 321 thumb; 400 mount surface

1. A cuff for a blood pressure information measuring device configured to be mounted on a measurement site for measuring blood pressure information comprising:
a fluid bag for compressing the measurement site;
a flexible curved elastic plate located outside said fluid bag while said fluid bag is wrapped around the measurement site, and annular or arc-shaped to extend along the measurement site;
an inflation and deflation mechanism secured to said curved elastic plate and inflating and deflating said fluid bag; and
a bag-shaped cover body encapsulating said fluid bag, said inflation and deflation mechanism, and said curved elastic plate.

2. The cuff for a blood pressure information measuring device according to claim 1, wherein
said inflation and deflation mechanism is disposed on a main surface of said curved elastic plate, and
said main surface is opposite to a main surface of said curved elastic plate that faces said fluid bag.

3. The cuff for a blood pressure information measuring device according to claim 1, further comprising a protection member covering an exposed surface of said inflation and deflation mechanism.

4. The cuff for a blood pressure information measuring device according to claim 1, wherein said inflation and deflation mechanism includes a pressurizing pump and an exhaust valve.

5. The cuff for a blood pressure information measuring device according to claim 1, further comprising:
a power supply unit supplying electric power for driving said inflation and deflation mechanism,
wherein said power supply unit is encapsulated in said bag-shaped cover body.

6. The cuff for a blood pressure information measuring device according to claim 1, further comprising:
a pressure detection unit for detecting an internal pressure of said fluid bag,
wherein said pressure detection unit is encapsulated in said bag-shaped cover body (12).

7. A blood pressure information measuring device comprising:
the cuff for the blood pressure information measuring device as recited in claim 1; and
an associated unit provided separately and independently of said cuff for the blood pressure information measuring device,
wherein said associated unit is provided with a display portion for displaying blood pressure information as a result of measurement.

8. The blood pressure information measuring device according to claim 7, wherein said associated unit is provided with an operation portion for entering an instruction to drive said inflation and deflation mechanism.

9. The blood pressure information measuring device according to claim 7, further comprising a communication unit enabling radio communication to be performed between said cuff for the blood pressure information measuring device and said associated unit.
